(19) United States
(12) Patent Application Publication
(10) Pub. No.: US 2009/0169466 A1
(43) Pub. Date: Jul. 2, 2009

(54) Methods of producing carbon nanotubes using peptide or nucleic acid micropatterning

(75) Inventors: Mineo Yamakawa, Campbell, CA (US); Yuegang Zhang, Cupertino, CA (US); Xing Su, Cupertino, CA (US); Lei Sun, Santa Clara, CA (US); Andrew A. Berlin, San Jose, CA (US); Narayanan Sundararajan, San Francisco, CA (US)

Correspondence Address: Client 21058;c/o DARBY & DARBY P.C., P.O. BOX 770, CHURCH STREET STATION, NEW YORK, NY, 10008-0770, US

(73) Assignee: INTEL CORPORATION

(21) Appl. No.: 11/344,712

(22) Filed: Jan. 31, 2006

Related U.S. Application Data
(62) Division of application No. 10/750,141, filed on Dec. 31, 2003.

(51) Int. Cl. D01F 9/12; C07H 21/04

(52) U.S. Cl. 423/447.1; 536/23.1; 977/742

(57) ABSTRACT

The methods, apparatus and systems disclosed herein concern ordered arrays of carbon nanotubes. In particular embodiments of the invention, the nanotube arrays are formed by a method comprising attaching catalyst nanoparticles 140, 230 to polymer 120, 210 molecules, attaching the polymer 120, 210 molecules to a substrate, removing the polymer 120, 210 molecules and producing carbon nanotubes on the catalyst nanoparticles 140, 230. The polymer 120, 210 molecules can be attached to the substrate in ordered patterns, using self-assembly or molecular alignment techniques. The nanotube arrays can be attached to selected areas 110, 310 of the substrate. Within the selected areas 110, 310, the nanotubes are distributed non-randomly. Other embodiments disclosed herein concern apparatus that include ordered arrays of nanotubes attached to a substrate and systems that include ordered arrays of carbon nanotubes attached to a substrate, produced by the claimed methods. In certain embodiments, provided herein are methods for aligning a molecular wire, by ligating the molecular wire to a double stranded DNA molecule.

### BACKGROUND OF THE INVENTION

1. Field of the Invention

The invention relates generally to carbon nanotube technology and more specifically to methods and systems for producing patterned arrays of carbon nanotubes.

2. Background Information

Carbon nanotubes can be thought of as sheets of graphite that have been rolled up into cylindrical tubes. The basic repeating unit of the graphite sheet consists of hexagonal rings of carbon atoms, with a carbon-carbon bond length of about 1.42 Å. Depending on how they are made, the tubes can be multiple walled or single walled.

The structural characteristics of nanotubes provide them with unique physical properties. Nanotubes can have up to 100 times the mechanical strength of steel and can be up to 2 mm in length. They exhibit the electrical characteristics of either metals or semiconductors, depending on the degree of chirality or twist of the nanotube. Carbon nanotubes have been used as electrical conductors and as electron field emitters. The electronic properties of carbon nanotubes are determined in part by the diameter and length of the tube.

Carbon nanotubes have become of increasing importance for the manufacture of microelectronic devices and microsensors. However, at present no method exists to efficiently produce ordered nanoscale or microscale assemblies of carbon nanotubes attached to areas 110, 310 of a substrate, where the distribution of nanotubes within an area 110, 310 is non-random. Using present methods, the distribution of nanotubes within each area 110, 310 of attachment to the substrate is essentially random. Such a random distribution can not provide optimal performance characteristics for various electrical and/or mechanical devices incorporating carbon nanotubes. Accordingly, there is a need for methods and systems for efficiently producing ordered nanoscale or microscale assemblies of carbon nanotubes attached to a substrate.

### BRIEF DESCRIPTION OF THE DRAWINGS

FIG. 1 illustrates an exemplary method for producing patterned arrays of carbon nanotubes using catalyst nanoparticles 140 attached to nucleic acids 120.

FIG. 2 illustrates an exemplary composition for producing patterned arrays of carbon nanotubes comprising catalyst nanoparticles 230 attached to peptides 210.

FIG. 3 illustrates an exemplary method for producing patterned arrays of carbon nanotubes using catalyst nanoparticles 230 attached to peptides 210.

FIG. 4 illustrates an exemplary method for fluidic alignment of single-stranded DNA.

### DETAILED DESCRIPTION OF THE INVENTION

As disclosed in more detail, provided herein is a method for producing carbon nanotubes that includes attaching one or more catalyst nanoparticles 140, 230 to one or more polymer 120, 210 molecules, attaching the polymer 120, 210 molecules to a substrate, typically removing the polymer 120, 210 molecules, and producing carbon nanotubes on the catalyst nanoparticles 140, 230. The polymer molecules 120, 210, can be, for example, a nucleic acid 120 or a peptide 210, which is optionally aligned before nanotubes are produced.

As used herein, “a’ or “an” can mean one or more than one of an item.

As used herein, the term “about” when applied to a number means within plus or minus ten percent of that number. For example, “about 100” means any number between 90 and 110.

“Nucleic acid” 120 encompasses DNA (deoxyribonucleic acid), RNA (ribonucleic acid), single-stranded, double-stranded or triple stranded and any chemical modifications thereof. The term also encompasses any known nucleic acid analog 120; including but not limited to peptide nucleic acids 120 (PNA), nucleic acid analog peptides (NAAP) 120 and locked nucleic acids 120 (LNA). A “nucleic acid” 120 can be of almost any length, from oligonucleotides 150 of 2 or more bases up to a full-length chromosomal DNA molecule. “Nucleic acids” 120 include, but are not limited to, oligonucleotides 150 and polynucleotides. Although nucleotide residues in naturally occurring nucleic acids 120 are typically joined together by phosphodiester bonds, within the scope of the disclosed methods nucleotide residues can be joined by phosphodiester bonds or by any other type of known covalent attachment.

The terms “protein” 210 “polypeptide” 210 and “peptide” 210 are used interchangeably herein to refer to polymeric molecules 120, 210 assembled from naturally occurring amino acids, non-naturally occurring amino acids, amino acid analogues and/or amino acid derivatives. The distinction between the terms is primarily one of length and the skilled artisan will realize that where the following disclosure refers to proteins 210, polypeptides 210 or peptides 210, the terms encompass polymers 120, 210 of any length. Although amino acid residues in naturally occurring proteins 210, polypeptides 210 and peptides 210 are typically joined together by peptide bonds, within the scope of the disclosed methods amino acid residues can be joined by peptide bonds or by any other type of known covalent attachment.

Carbon nanotubes have strong electronic properties that are modulated by the length and diameter of the tube. A simple estimate of the effect of tube length on electronic wave function is given by:

ΔE=hvF/2L

Where ΔE represents energy level splitting, L is tube length, h is Planck's constant and vF is the Fermi velocity (8.1×10⁵ m/sec) (Venema et al., “Imaging Electron Wave Functions of Carbon Nanotubes,” Los Alamos Physics Preprints:cond-mat/9811317, 23 Nov. 1996.) The difference between electron energy levels is inversely proportional to the length of the nanotube, with finer splitting observed for longer tubes.

The electronic properties of carbon nanotubes are also a function of tube diameter. The relationship between fundamental energy gap (highest occupied molecular orbital−lowest unoccupied molecular orbital) and tube diameter can be modeled by the function.

E_{gap}=2y₀a_{cc}/d

Where y₀ is the carbon-carbon tight bonding overlap energy (2.7±0.1 eV), a_{cc} is the nearest neighbor carbon-carbon distance (0.142 nm) and d is the tube diameter (Jeroen et al., Nature 391:59-62, 1998). As energy is increased over the Fermi energy level, sharp peaks in the density of states, referred to as Van Hove singularities, appear at specific energy levels (Odom et al., Nature 391:62-64, 1998).

In certain embodiments of the invention, nanotubes can have lengths of about 10 to 100 nm, 100 to 200 nm, 200 to 500 nm, 500 nm to 1 μm, 1 to 2 μm, 2 to 5 μm, 5 to 10 μm, 10 to 20 μm, 20 to 50 μm and/or 50 to 100 μm. In other embodiments, longer nanotubes of up to 1-2 mm in length can be used. In some embodiments, single walled carbon nanotubes with a diameter of about 1 to 1.5 nm can be used. In other embodiments, nanotubes diameters of about 1 to 2 nm, 2 to 3 nm, 1 to 5 nm and/or 2-10 nm can be used. The length and/or diameter of the nanotubes to be used are not limited and nanotubes of virtually any length or diameter are contemplated, including single-walled and double-walled nanotubes. In particular embodiments of the invention, nanotube diameter and length can be selected to fall within particular size ranges. As discussed below, nanotube diameter can be determined, at least in part, by the size of the catalyst nanoparticles 140, 230 used. A variety of methods for controlling nanotube length are known (e.g., U.S. Pat. No. 6,283,812) and any such known method can be used.

Particular embodiments disclosed herein, involve methods for producing and/or apparatus including patterned nanotube arrays attached to a substrate. In various embodiments, the average distance between nanotubes, the range of nanotube distances or even the specific pattern of nanotube distribution on the substrate can be controlled. Such nanotube arrays are of use for a variety of applications, including, but not limited to, fabrication of miniature electronic, chemical and molecular devices, probes for use in scanning probe microscopy, molecular wires, incorporation into ultrafast random access memory (Rueckes et al., Science 289:94, 2000), field-effect transistors, single electron transistors, field emitter arrays, flat screen panels, electromechanical transducers, molecular switches and any other known use for carbon nanotube arrays.

A variety of methods for production of carbon nanotubes are known, including carbon-arc discharge, chemical vapor deposition via catalytic pyrolysis of hydrocarbons, plasma assisted chemical vapor deposition, laser ablation of a catalytic metal-containing graphite target and condensed-phase electrolysis. (See, e.g., U.S. Pat. Nos. 6,258,401, 6,283,812 and 6,297,592.) However, such known methods do not result in nanotubes attached to substrates in precisely patterned arrays.

In various embodiments of the present invention, patterned arrays of carbon nanotubes attached to substrates can be produced, using catalyst nanoparticles 140, 230 attached to a polymer 120, 210, such as a nucleic acid 120 or peptide 210. Because the polymer 120, 210 molecules can be attached to a substrate in an ordered pattern before nanotube synthesis, the resulting nanotubes become attached to the substrate in an ordered pattern, determined by the distribution of catalyst containing polymer 120, 210 molecules on the substrate. Before nanotube production, the polymer 120, 210 molecules can be removed, for example by heating to about 600 to 800° C. in air or oxygen.

Methods of carbon nanotube production using catalyst nanoparticles 140, 230, such as ferritin, are known. (See, e.g., Dai, Acc. Chem. Res. 35:1035-44, 2002; Kim et al., Nano Letters 2:703-708, 2002; Bonard et al., Nano Letters 2:665-667, 2002; Zhang et al., Appl. Phys. A 74:325-28, 2002; U.S. Pat. Nos. 6,232,706 and 6,346,189). Typically, catalyst nanoparticles 140, 230 are used in combination with chemical vapor deposition (CVD) techniques, by flowing a hydrocarbon gas (e.g., CH₄, C₂H₄) through a catalyst-containing tube reactor at temperatures of about 500 to 1000° C., using H₂ gas co-flow to provide reducing conditions. The catalyst nanoparticles 140, 230 serve as nucleation sites for carbon nanotube formation and growth. Under such conditions, the diameter of the nanotube formed appears to be a function of the diameter of the catalyst nanoparticle 140, 230 used (Dai, 2002). It has been suggested that the mechanism of nanotube formation involves absorption of decomposed carbon atoms into the nanoparticle 140, 230 to form a solid-state carbon-metal solution, followed by supersaturation and precipitation of the carbon atoms out from the nanoparticle 140, 230 and their incorporation into the base of the growing nanotube (Dai, 2002).

To further control the arrangement of the nanotube array, carbon nanotubes can be grown by CVD techniques in the presence of an external electrical field, using one or more pairs of microfabricated electrodes attached to a substrate, with a field intensity of about 1 to 5 V/μm (volt per micrometer) (e.g., Dai, 2002). The electrical field induces a dipole in the growing single-wall carbon nanotubes (SWNTs) parallel to their long axis, forcing the nanotubes to grow parallel to the electrical field. In various embodiments, nanotubes can be aligned at angles to each other, using two or more pairs of electrodes with differently oriented electrical fields. Nanotube alignment by electrical field is reported to be stable against thermal fluctuations at the temperatures used for CVD growth (Dai, 2002).

Such methods have been used to produce arrays of carbon nanotubes attached to a substrate, such as a silicon chip, wherein the areas 110, 310 in which nanotubes are formed can be determined by controlling the distribution of catalyst nanoparticles 140, 230 on the substrate, for example by standard photo- or electron-beam lithography, shadow masking or microcontact printing (Bonard et al., 2002). However, the pattern of nanotube distribution within each such area 110, 310 on the substrate is essentially random, with little or no control over the nanotube-to-nanotube distance or the precise pattern of nanotube distribution within each area 110, 310. Using the methods disclosed herein, it is possible to determine the distances between adjacent nanotubes and control the patterns of nanotube distribution within individual areas 110, 310 on the substrate, by attaching catalyst nanoparticles 140, 230 to one or more selected locations on a polymer 120, 210, such as a protein 210, peptide 210 or nucleic acid 120. Because the polymers 120, 210 themselves can be induced to pack together in an ordered pattern on the substrate, for example by using a viral coat protein polymer 210 or by using nucleic acids 120 or peptides 210 of known configuration in combination with a molecular alignment technique, it is possible to produce arrays of carbon nanotubes wherein the spacing and distribution of nanotubes within each selected area 110, 310 on the chip can be determined.

A number of known techniques for molecular alignment of polymer 120, 210 molecules can be of use, including but not limited to use of optical tweezers (e.g. Walker et al., FEBS Lett. 459:39-42, 1999; Smith et al., Am. J. Phys. 67:26-35, 1999), direct current (DC) and/or alternating current (AC) electrical fields (e.g., Adjari and Prost, Proc. Natl. Acad. Sci. U.S.A. 88:4468-71, 1991), magnetic fields with ferromagnetic nanoparticles 140, 230, microfluidic (hydrodynamic) flow and/or molecular combing (e.g., U.S. Pat. Nos. 5,840,862; 6,054,327; 6,344,319). The method of alignment is not limiting and any known method can be used. Techniques for molecular alignment of polymer 120, 210 molecules attached to the substrate can be used in combination with techniques for aligning carbon nanotubes, as discussed above.

The attachment sites for catalyst nanoparticles 140, 230 on individual polymer 120, 210 molecules can be determined. For example, streptavidin modification of specific amino acid residues on a protein 210 or peptide 210 can be used to bind biotinylated ferritin 140, 230 to selected sites on the three-dimensional protein 210 or peptide 210 structure. Alternatively, streptavidin-modified oligonucleotide 150 probes can be used to hybridize to selected locations on a single-stranded DNA molecule 120, followed by binding of biotinylated ferritin 140, 230. Many techniques for site-specific modification of proteins 210, peptides 210, nucleic acids 120 and other polymers 120, 210 are known and can be used in the disclosed methods. For example, peptides 210 or nucleic acids 120 can be chemically synthesized, incorporating modified amino acids (e.g., biotinylated lysine or biocytin 220) or modified nucleotides into the growing polymer 120, 210 at predetermined locations within the polymer 120, 210 sequence. The modified amino acid or nucleotide residues can then be used to attach catalyst nanoparticles 140, 230 to specific locations on the polymer 120, 210. Analogues of amino acids or nucleotides can also be used for site-specific attachment of nanoparticles 140, 230. Alternatively, specific types of residues, such as cysteine or lysine residues in proteins 210 or peptides 210, can be chemically modified after synthesis using standard techniques. The modified amino acid residues can then serve as attachment sites for catalyst nanoparticles 140, 230. In other alternatives, side-chain specific reagents can be used to create nanoparticle 140, 230 binding sites. For example, biotin-PE-maleimide (Dojindo Molecular Technologies, Inc., Gaithersburg, Md.) can be reacted with cysteine residues of proteins 210 or peptides 210 or with sulfhydryl modified nucleotides. The biotin moiety 160 can then be used to attach an avidin-ferritin conjugated nanoparticle 140, 230.

Although proteins 210, peptides 210 and single-stranded nucleic acids 120 are shown in exemplary embodiments of the invention disclosed herein, the embodiments are not limited to any specific form of polymer 120, 210. In alternative embodiments it is possible to bind modified oligonucleotides 150 to a double-stranded nucleic acid 120 to form short segments of triple-stranded structure that can bind to catalyst nanoparticles 140, 230. Alternatively, other types of known polymers 120, 210 besides nucleic acids 120, peptides 210 and proteins 210 can be used for nanoparticle 140, 230 attachment. Such polymers 120, 210 can include, but are not limited to, lipids, polysaccharides, glycolipids, glycoproteins, lipopolysaccharides, lipoproteins, alkanes, alkenes, alkynes, fatty acids, phospholipids, sphingolipids, etc. In certain embodiments, branched polymers 120, 210 such as branched nucleic acids 120 or branched proteins 210 can be used.

Protein-coated iron nanoparticles 140, 230, such as ferritin, are commercially available, including as conjugates of biotin 160 or avidin 170 (e.g, Vector Laboratories, Burlingame, Calif.; E-Y Laboratories, Inc., San Mateo, Calif.), suitable for attachment to polymer 120, 210 molecules. Alternatively, nanoparticles 140, 230 of defined size can be made by known methods (e.g., Li et al. J. Phys. Chem. B, 105:11424-431, 2001). For example, controllable numbers of Fe³⁺ atoms can be inserted into the cores of apoferritin (Zhang et al., 2002). Calcination in air, for example at 800° C. for 5 min, removes the ferritin shell and oxidizes the iron core, resulting in the production of discrete Fe²O³ nanoparticles 140, 230 of about 1.5 nm average size that are suitable for catalytic growth of SWNTs (Dai, 2002). The type of nanoparticle 140, 230 used is not limiting. Although the disclosed methods concern the use of iron-containing ferritin nanoparticles 140, 230, other known types of catalyst nanoparticles 140, 230 such as non-ferritin iron nanoparticles 140, 230, nickel nanoparticles 140, 230, cobalt nanoparticles 140, 230, molybdenum nanoparticles 140, 230, zinc nanoparticles 140, 230, ruthenium nanoparticles 140, 230 and/or alloy nanoparticles 140, 230 can be used. The only requirement is that the catalyst nanoparticle 140, 230 be capable of catalyzing carbon nanotube formation.

As indicated herein, typically during nanotube production, polymer molecules are removed. However, in certain aspects of the methods disclosed herein, the catalyst is molybdenum nanoparticles 140, 230 and the polymer 120, 210 molecules are not removed during nanotube production.

In one embodiment, the present invention provides arrays of carbon nanotubes produced using catalyst nanoparticles attached to nucleic acids 120. Nucleic acid molecules 120 of use can be prepared by any known technique. In one embodiment of the invention, the nucleic acids 120 can be naturally occurring single- or double-stranded DNA molecules. Methods for preparing and isolating various forms of cellular nucleic acids 120 are known (See, e.g., Guide to Molecular Cloning Techniques, eds. Berger and Kimmel, Academic Press, New York, N.Y.; 1987; Molecular Cloning: A Laboratory Manual, 2nd Ed., eds. Sambrook, Fritsch and Maniatis, Cold Spring Harbor Press, Cold Spring Harbor, N.Y., 1989). Where appropriate, naturally occurring nucleic acids 120 can be restricted and sorted into shorter length fragments using known techniques, for example, restriction endonuclease digestion and gel electrophoresis or high pressure liquid chromatography (HPLC). In aspects where double-stranded nucleic acids 120 are prepared, the nucleic acids 120 are typically burned off and optionally denatured before carbon nanotubes are formed from catalyst nanoparticles 140, 230 attached to the nucleic acids or attached to oligonucleotides that hybridize to the nucleic acids 120.

Naturally occurring nucleic acids 120 can be single or double-stranded. Where double-stranded nucleic acids 120 are used, the strands can be separated using known techniques, for example heating to about 95° C. for about 5 minutes, to separate the two strands, either before or after attachment to the substrate. Single-stranded nucleic acids 120 can be used to facilitate hybridization to specific probe sequences; such as biotin 160 conjugated oligonucleotides 150.

Naturally occurring nucleic acids 120 or fragments thereof can be of any selected length. In certain embodiments of the invention, nucleic acids 120 of up to about 10,000 basepairs (10 kb), or about 3.4 μm in length can be used. Naturally occurring nucleic acids 120 of greater length, up to full-length chromosomal DNA, are known and can be used in the disclosed methods. Where a highly reproducibly sized DNA fragment 120 is needed, a plasmid, cosmid, bacterial chromosome or other natural nucleic acid 120 of known size can be replicated, purified and, for example, cut with a known single-site restriction endonuclease to produce double-stranded nucleic acids 120 of precise size.

In other embodiments of the invention, non-naturally occurring nucleic acids 120 can be used. For example, double-stranded nucleic acids 120 can be prepared by standard amplification techniques, such as polymerase chain reaction (PCR3) amplification. Amplification can utilize primer pairs designed to bind to a template and produce amplified segments (amplicons) of any selected size, up to thousands of base-pairs in length. Methods of nucleic acid 120 amplification are well known in the art.

Other sources of non-naturally occurring nucleic acids 120 include chemically synthesized nucleic acids 120. Such nucleic acids 120 can be obtained from commercial sources (e.g., Midland Certified Reagents, Midland Tex.; Proligo, Boulder, Colo.). Alternatively, nucleic acids 120 can be chemically synthesized using a wide variety of oligonucleotide 150 synthesizers that can be purchased from commercial vendors (e.g., Applied Biosystems, Foster City, Calif.). Typically, chemically synthesized nucleic acids 120 are of somewhat limited size. After about fifty to one hundred nucleotides have been incorporated, the efficiency of incorporation results in low yields of product. However, shorter oligonucleotides 150 can be increased in length, for example by hybridization of overlapping complementary sequences followed by ligation. Chemical synthesis of nucleic acids 120 allows the incorporation of modified nucleotides or nucleotide analogues that can be incorporated at any selected site in the nucleic acid 120 sequence and can serve as attachment sites for catalyst nanoparticles 140, 230. In alternative embodiments of the invention, nanoparticle 140, 230 attachment-sites can be located using hybridization with modified oligonucleotides 150. Such oligonucleotides 150 can be designed to bind to only one site on a nucleic acid 120 sequence and can be modified, for example by biotinylation, to facilitate attachment of nanoparticles 140, 230, such as avidin-ferritin nanoparticles 140, 230.

In various embodiments of the invention, nucleic acid molecules 120 can be immobilized by attachment to a solid surface. Immobilization of nucleic acid molecules 120 can be achieved by a variety of known methods involving either non-covalent or covalent attachment. For example, immobilization can be achieved by coating a solid surface with streptavidin or avidin 170 and binding of a biotin 160 conjugated nucleic acid 120. Immobilization can also occur by coating a silicon, quartz, polymeric surface such as PDMS (polydimethyl siloxane) or other solid surface with poly-L-Lys or aminosilane, followed by covalent attachment of either amino- or sulfhydryl-modified nucleic acids 120 using bifunctional crosslinking reagents. Bifunctional cross-linking reagents of potential use include glutaraldehyde, bifunctional oxirane, ethylene glycol diglycidyl ether, and carbodiimides, such as 1-ethyl-3-(3-dimethylaminopropyl) carbodiimide.

Immobilization can take place by direct covalent attachment of 5′-phosphorylated nucleic acids 120 to chemically modified surfaces, for example acid treated silicon. The covalent bond between the nucleic acid 120 and the solid surface can be formed by condensation with a cross-linking reagent. This method facilitates a predominantly 5′-attachment of the nucleic acids 120 via their 5′-phosphates.

Nucleic acids 120 can be bound to a surface by first silanizing the surface, then activating with carbodiimide or glutaraldehyde. Alternative procedures can use reagents such as 3-glycidoxypropyltrimethoxysilane or aminopropyltrimethoxysilane (APTS) with nucleic acids 120 linked via amino linkers incorporated either at the 3′ or 5′ end of the molecule during DNA synthesis. Other methods of immobilizing nucleic acids 120 are known and can be used.

In certain aspects of the invention a capture oligonucleotide 150 can be bound to a surface. The capture oligonucleotide 150 will hybridize with a specific sequence of a nucleic acid 120 attached to a catalyst nanoparticle 140, 230. In alternative aspects, following nucleic acid 120 hybridization to a capture oligonucleotide 150, a set of oligonucleotides 150 labeled with catalyst nanoparticles 140, 230 can be hybridized to the bound nucleic acid 120.

The type of surface to be used for immobilization of the nucleic acid 120 is not limited. In various embodiments, the immobilization surface can be quartz, silicon, silicon oxide, silicon dioxide, silicon nitride, germanium, or any other surface known in the art, so long as the surface is stable to the application of temperatures that can reach as high as 1000° C. during carbon nanotube formation.

In some embodiments of the invention, nucleic acids 120 or other polymer 120, 210 molecules can be aligned on a substrate prior to synthesis of carbon nanotubes. The nucleic acids 120 can first be attached to specific areas 110, 310 on the substrate using known techniques. For example, the substrate can be patterned with a thin film of gold, using photo- or electron-beam lithography, shadow masking or microcontact printing (e.g., Bonard et al., 2002). Thiol-modified nucleic acids 120 can be covalently bonded to the gold patches 110, 310 on the substrate. Methods for attaching proteins 210, nucleic acids 120 and other polymers 120, 210 to specific areas 110, 310 of a substrate are well known and any such known method can be used, including but not limited to photolithography and etching, laser ablation, molecular beam epitaxy, dip-pen nanolithography, chemical vapor deposition (CVD) fabrication, electron beam or focused ion beam technology or imprinting techniques.

The attached nucleic acids 120 can be aligned using any of a number of known techniques. An exemplary method for aligning nucleic acids 120 on a substrate is known as molecular combing. (See, e.g., Bensimon et al., Phys. Rev. Lett. 74:4754-57, 1995; Michalet et al., Science 277:1518-23, 1997; U.S. Pat. Nos. 5,840,862; 6,054,327; 6,225,055; 6,248,537; 6,265,153; 6,303,296 and 6,344,319.) In this technique, nucleic acids 120 or other hydrophilic polymers 120, 210 are attached at one or both ends to a substrate, such as a silicon chip. The substrate and attached nucleic acids 120 are immersed in a solution, such as an aqueous buffer, and slowly withdrawn from the solution. The movement of the air-water-substrate interface serves to align the attached nucleic acids 120, parallel to the direction of movement of the meniscus.

The method of polymer 120, 210 alignment used is not limiting and any known method, including but not limited to use of optical tweezers, DC and/or AC electrical fields, microfluidic flow, and/or magnetic fields applied to attached ferromagnetic nanoparticles 140, 230 is contemplated. In another non-limiting example, nucleic acids 120 or other charged polymers 120, 210 can be aligned on a substrate by free flow electrophoresis (e.g., Adjari and Prost, Proc. Natl. Acad. Sci. U.S.A. 88:4468-71, 1991). The surface can comprise alternating bands of conductive and non-conductive materials that function as electrodes, or other types of microelectrodes can be used. In the presence of an alternating current electrical field, polymers 120, 210 comprising charged residues, such as the phosphate groups on nucleic acids 120, will align with the field (Adjari and Prost, 1991). The method is not limited to nucleic acids 120 and can be applied to proteins 210 or other polymers 120, 210 containing charged groups. Where the charge on the polymer 120, 210 is not fixed, the net charge can be manipulated, for example by changing the pH of the solution.

Fluidic alignment of various types of polymer molecules (i.e. molecular wires or concatenated molecular chains), has been demonstrated (Bensimon et al., Science, 265: 1096-98 (1994) (double stranded DNA); Lieber et al., Science, 291:630 (2001) (semiconductor nanowires); Lienemann et al., Nanoletters, 1:345 (2001) (single-stranded DNA)). However, one problem with these methods, is the low alignment yield for short molecular wires. Single stranded DNA are especially hard to align for the following reasons:

1.) The flow often does not provide enough dragging force to break the intramolecular base-pairing (Hansma, et al., Nucleic Acids Res. 24:713 (1996));

2.) Single-stranded nucleic acids are very flexible, making it difficult to keep them from relaxing after drying;

3.) Some molecules attach to a highly positively-charged surface, before being aligned; and

4.) Atomic force microscopy (AFM) observation of single-stranded nucleic acids is difficult due to their short height.

To attempt to solve these problems, Lienemann et al. (2001) heated DNA before fluidic alignment to break up the intramolecular base-pairing. Although this achieved moderate success in alignment yield, the heating step denatured any features on the nucleic acid that are attached via hybridization. Therefore, applications such as nucleic acid-directed patterning are not possible with this method.

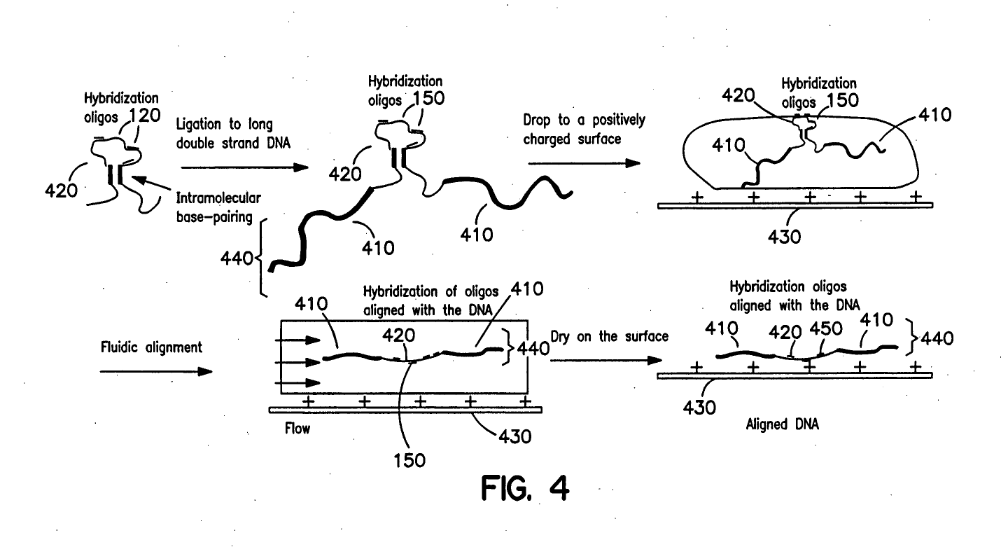

Accordingly, provided herein is a method to align short molecular wires 420 with high yield without heat denaturation, as shown in FIG. 4. According to this method, double-stranded DNA 410, such as phage E DNA, is attached to both ends of a molecular wire 420, and fluidic alignment is performed on an anchor surface. The anchor surface in certain examples, is a positively-charged surface 430. This method is referred to herein as, inter alia, “double-stranded DNA/forced flow alignment.”

The method for aligning a molecular wire 420 includes ligating the molecular wire 420 to a double stranded DNA molecule 410 to create a double-stranded DNA/molecular wire hybrid molecule 440, which is applied to a positively charged surface 430, and aligned to the positively charged surface 430 using fluidic alignment. Furthermore, the method typically involves drying the double-stranded DNA/molecular wire hybrid molecule 440 to the surface 430. The molecular wire 420 is “sandwiched” between two double-stranded nucleic acids 410 in the double-stranded DNA/molecular wire hybrid molecule 440.

In certain aspects, the molecular wire 420 is a single-stranded nucleic acid 120. In other aspects, the molecular wire is a peptide. In certain aspects, for example, the molecular wire 420 includes a catalyst nanoparticle 140, 230, such as a ferritin nanoparticle, that is bound directly or indirectly, or includes a binding partner, such as biotin or avidin to which a catalyst nanoparticle can be bound. Therefore, in certain aspects the molecular wire 420 is a single-stranded nucleic acid molecule 120, such as single-stranded DNA, that is attached to a catalytic nanoparticle 140, 230. Furthermore, the method can include producing carbon nanotubes on the catalyst nanoparticles 140, 230.

In certain aspects, an oligonucleotide 150 is bound to a single-stranded nucleic acid molecule 120 molecular wire 420 that is sandwiched between double-stranded DNA 410 on the double-stranded DNA/molecular wire hybrid molecule 440. For example, the oligonucleotide 150 can be a modified oligonucleotide 150, or a population of modified oligonucleotides 150, that are hybridized to the single-stranded DNA 120. Furthermore, the modified oligonucleotide 150, 460 or population of modified oligonucleotides 150, 460, can be modified by attachment to a catalytic nanoparticle 140, such as ferritin, directly or indirectly, as disclosed in more detail hereinbelow. In these aspects, the single-stranded DNA 120 sandwiched between double-stranded DNA 410 on the double-stranded DNA/molecular wire hybrid molecule 440, is a capture oligonucleotide 120 as disclosed hereinbelow that hybridizes to the modified oligonucleotides 150, 460. The modified oligonucleotide 150, for example, can be modified with a biotin moiety that is linked to a catalytic nanoparticle 140 via an avidin moiety.

A double stranded DNA 120 that is used in the double-stranded DNA/forced flow alignment methods provided herein, is not limited with regard to a specific nucleotide sequence, but is typically between about 100 and 1,000,000 nucleotides in length, in certain aspects between 500 and 50,000 nucleotides in length. In certain aspects, the double-stranded DNA is phage lambda DNA. Methods for ligating double-stranded DNA to molecular wires such as single-stranded DNA and peptides are known in the art. For example, DNA ligase can be used to ligate double-stranded DNA to single-stranded DNA.

Methods provided herein for double-stranded DNA/forced flow alignment provide much larger stretching force on the molecular wires, such as single-stranded DNA, that is created on the double-stranded DNA and passed on to the ligated molecular wire. Therefore, steps such as heat denaturation can be avoided. Furthermore, after drying, the double-stranded DNA attaches to the surface firmly and serves as an anchor such that molecular wires that are bound on each end by double-stranded DNA will maintain their linear confirmation. In addition, less positively charged surfaces are necessary for alignment, further enhancing alignment yield. Finally, long double-stranded DNA is easy to visualize using AFM or fluorescence microscopy. This allows visualization of the molecular wire such as single-stranded DNA, by following the double-stranded DNA.

As will be understood, many different positively charged surfaces can be employed for double-stranded DNA/forced flow alignment. For example, the immobilization surface can be quartz, silicon, silicon oxide, silicon dioxide, silicon nitride, germanium, or any other surface known in the art, so long as the surface is positively charged and stable to the application of temperatures that can reach as high as 1000° C. during carbon nanotube formation.

In one aspect, of a method herein, circular M13 DNA is cut by a restriction enzyme to form a single stranded DNA and hybridized with biotin-labeled short strands specific to particular sequences of the m13 DNA. The M13 DNA is then ligated on either side to lambda-phage DNA. The biotin labels are then used to attach avidin-ferritin molecules.

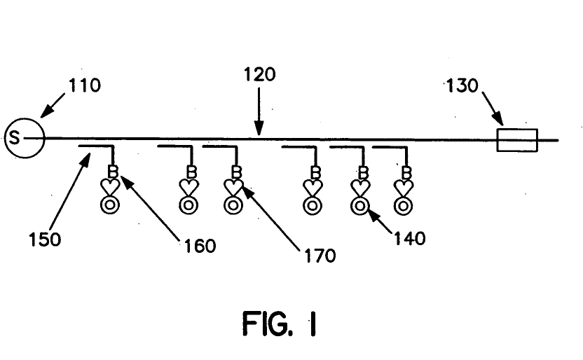

A number of techniques can be used to attach catalyst nanoparticles to aligned or non-aligned nucleic acids. An exemplary embodiment of the invention, illustrating a method for producing patterned arrays of carbon nanotubes using nucleic acids 120 attached to a substrate, is disclosed in FIG. 1. A nucleic acid 120 attachment area 110 on the substrate, such as a gold patch 110, is used to attach nucleic acid polymers 120. The attachment areas 110 can be anywhere from 1 nm to about 100 nm in size or greater, up to 1 μm in size. For certain applications, attachment areas 110 of greater than 1 μm in size can be used. Depending on the application, the substrate structures to which nanotubes are attached can be comprised of conductive and/or nonconductive materials, as are well known in the art.

In the example illustrated in FIG. 1, the polymer 120 is a single-stranded DNA molecule. One end of the polymer 120 can be covalently modified, for example with a thiol group, for attachment to the DNA binding areas 110 on the substrate. The DNA molecules 120 attached to the substrate can be aligned, for example using optical tweezers, molecular combing, magnetic fields, microfluidic flow and/or free-flow electrophoresis. In particular embodiments of the invention, the other end of the nucleic acid 120 can be modified with a second group 130 to anchor the DNA 120 to the substrate after alignment. Alternatively, the DNA molecules 120 can be immobilized by applying positive charges to the substrate and drying the DNA molecules 120 on the substrate. In certain aspects, the DNA molecules 120 are aligned using double-stranded DNA/forced flow alignment, as disclosed herein. Other known methods of attaching nucleic acids 120 to substrates, discussed above, can be used.

In some embodiments of the invention, streptavidin-coated microbeads can be used to identify and/or quantify DNA molecules 120. The number of DNA molecules 120 attached to an area 110 can be quantified, for example, by measuring the spring tension of a DNA-bead complex or by visualizing dye-stained DNA molecules 120. In certain embodiments, it is possible to have a single DNA molecule 120 attached to a gold patch 110.

As shown in FIG. 1, catalyst nanoparticles 140 can be attached to the DNA polymer 120 using hybridization with modified oligonucleotides 150. The sequences of the oligonucleotides 150 can be designed to bind to only one complementary sequence within each DNA polymer 120, or can be designed to bind to multiple sites on each DNA molecule 120. The positions and distances between adjacent oligonucleotides 150 can be selected by choosing the appropriate complementary sequences for hybridization.

In this exemplary embodiment, the oligonucleotides 150 are conjugated to biotin moieties 160 at one end. To facilitate nanoparticle 140 binding, the sequence of the biotin 160 labeled end of the oligonucleotide 150 can be designed so that it is not complementary to the DNA molecule 120. Thus, the biotin 160 labeled end of the oligonucleotide 150 will stick out from the surface of the substrate. This facilitates non-covalent binding of the biotin 160 labeled end, for example, to a catalyst nanoparticle 140 conjugated to an avidin moiety 170. Because the binding interaction occurs with a one-to-one stoichiometry, each oligonucleotide 150 will attach only one catalyst nanoparticle 140. In this non-limiting example, each catalyst nanoparticle 140 comprises an avidin 170 conjugated ferritin molecule 140. Non-hybridized oligonucleotides 150 and non-conjugated nanoparticles 140 can be washed off the substrate, for example using an aqueous buffer with a non-ionic surfactant. The distribution of nanoparticles 140 on the substrate can be verified by scanning electron microscopy (SEM), transmission electron microscopy (TEM), scanning probe microscopy (SPM) or other known techniques.

The skilled artisan will realize that the disclosed embodiment of the invention is not limiting and other techniques for attaching nucleic acids 120 to substrates and/or attaching catalyst nanoparticles 140 to the nucleic acids 120 can be utilized. In some cases, the nucleic acid 120 can be directly modified to bind ferritin 140, for example by incorporation of biotin 160 labeled nucleotides directly into the DNA molecule 120. In alternative embodiments of the invention, use of a linking group, such as an oligonucleotide 150, can facilitate nanoparticle 140 binding by decreasing steric hindrance.

Once catalyst nanoparticles 140 are attached to the substrate, carbon nanotubes can be grown on the nanoparticles 140 using CVD techniques as disclosed above. Following nanotube synthesis, the remaining DNA molecules 120 can be removed from the substrate by, for example, heating in air or oxygen to about 600 to 800° C., leaving an ordered array of iron oxide nanotubes attached to the substrate.

In the following discussion, the terms “protein” 210 and “proteins” 210 are used to refer to amino acid polymers 210 of any length, including peptides 210, polypeptides 210 and proteins 210.

In another embodiment, provided herein are methods for producing arrays of carbon nanotubes using catalyst nanoparticles attached to peptides or proteins. Purified proteins 210 can be purchased from a wide variety of commercial sources, such as Sigma Chemicals (St. Louis, Mo.), Bio-Rad Laboratories (Hercules, Calif.), Promega (Madison, Wis.) and many other companies. Proteins 210 can also be purified from a variety of sources, using techniques well known in the art. Such techniques typically involve an initial crude fractionation of cell or tissue homogenates and/or extracts into protein 210 and non-protein fractions. Fractionation can utilize, for example, differential solubility in aqueous solutions, detergents and/or organic solvents, elimination of contaminants by enzymatic digestion, precipitation of proteins 210 with ammonium sulphate, polyethylene glycol, antibodies, heat denaturation and the like, followed by ultracentrifugation. Low molecular weight contaminants can be removed by dialysis, ultrafiltration and/or organic phase extraction.

Proteins 210 can be further purified using chromatographic and/or electrophoretic techniques including, but not limited to, ion-exchange chromatography, gel exclusion chromatography, polyacrylamide gel electrophoresis, affinity chromatography, immunoaffinity chromatography, hydroxylapatite chromatography, hydrophobic interaction chromatography, reverse phase chromatography, isoelectric focusing, fast protein liquid chromatography (FPLC) and high pressure liquid chromatography (HPLC). Immunoaffinity chromatography and other immunology-based techniques rely upon the use of monoclonal or polyclonal antibodies specific for the protein 210 of interest. Such antibodies can be commercially purchased or can be prepared using standard techniques known in the art (e.g., Harlow and Lane, Antibodies: A Laboratory Manual, Cold Spring Harbor Laboratory, Cold Spring Harbor, N.Y., 1988).

In alternative embodiments of the invention, proteins 210 can be expressed using an in vitro translation system with an mRNA template. Kits for performing in vitro translation are available from commercial sources, such as Ambion (Austin, Tex.), Promega (Madison, Wis.), Amersham Pharmacia Biotech (Piscataway, N.J.), Invitrogen (Carlsbad, Calif.) and Novagen (Madison, Wis.). Such kits can utilize total RNA, purified polyadenylated mRNA, and/or purified individual mRNA species. Commonly used in vitro translation systems are based on rabbit reticulocyte lysates, wheat germ extracts or E. coli extracts. The systems contain crude cell extracts including ribosomal subunits, transfer RNAs (tRNAs), aminoacyl-tRNA synthetases, initiation, elongation and termination factors and/or all other components required for translation. In certain embodiments of the invention, the natural amino acids present in such extracts can be supplemented with one or more different types of labeled amino acids, such as biocytin 220.

In certain alternative embodiments of the invention, in vitro translation can be linked to transcription of genes to generate mRNAs. Such linked transcription/translation systems can use PCR® amplification products and/or DNA sequences inserted into standard expression vectors such as BACs (bacterial artificial chromosomes), YACs (yeast artificial chromosomes), cosmids, plasmids, phage and/or other known expression vectors. Linked transcription/translation systems are available from commercial sources (e.g., Proteinscript3 II kit, Ambion, Austin, Tex.; Quick Coupled System, Promega, Madison, Wis.; Expressway, Invitrogen, Carlsbad, Calif.).

Nucleic acids 120 encoding proteins 210 of interest can also be incorporated into expression vectors for transformation into host cells and production of the encoded proteins 210. A complete gene can be expressed or fragments of a gene encoding portions of a protein 210 can be expressed. The gene or gene fragment encoding protein(s) 210 of interest can be inserted into an expression vector by standard cloning techniques.

In other embodiments of the invention, the proteins 210 to be used can be prepared by chemical synthesis. Various automated protein 210 synthesizers are commercially available and can be used in accordance with known protocols. (See, for example, Stewart and Young, Solid Phase Peptide Synthesis, 2d ed., Pierce Chemical Co., 1984; Tam et al., J. Am. Chem. Soc., 105:6442, 1983; Merrifield, Science, 232:341-347, 1986; Barany and Merrifield, The Peptides, Gross and Meienhofer, eds., Academic Press, New York, pp. 1-284, 1979.) Short protein 210 sequences, usually up to about 50 to 100 amino acids in length, can be readily synthesized by such methods. Such synthetic proteins 210 can be designed to contain modified amino acid residues and/or amino acid analogues at specific locations within the protein 210 sequence. Longer synthetic proteins 210 can be prepared by chemically synthesizing and purifying shorter fragments and covalently cross-linking the fragments together, for example by carbodiimide catalyzed formation of peptide bonds. However, longer proteins 210 are typically prepared by cloning an appropriate nucleic acid 120 sequence encoding the protein 210 of interest into an expression vector as discussed above. In various embodiments of the invention, proteins 210 of up to about 100 amino acid residues in length (about 20 to 40 nm in size) can be used. In other embodiments, proteins 210 of any length between 10 amino acid residues up to full-length proteins 210 of thousands of amino acid residues can be used.

In some embodiments of the invention, synthetic proteins 210 to be used can be designed to exhibit particular three-dimensional structures and/or to spontaneously assemble into ordered quaternary aggregates of proteins 210 (e.g., Aggeli et al., Proc. Natl. Acad. Sci. USA, 98:11857-11862, 2001; Brown et al., J. Am. Chem. Soc., 124:6846-48, 2002). The effect of primary protein 210 structure (amino acid sequence) on secondary and tertiary structure is known in the art.

Computer modeling of protein 210 structure has been used to predict types of secondary structure, such as alpha helices, beta sheets and reverse turns, based upon empirical rules such as those proposed by Chou and Fasman (Adv. Enzymol. 47:45-148, 1978). Each type of amino acid residue is assigned a probability value of forming different types of secondary structure and a moving window algorithm looks for regions of probable structure. Where de novo protein 210 synthesis is used, particular types of secondary structures, such as alpha helices, can be designed by incorporating a high percentage of alpha-helix forming residues. The ends of helices can be designed by incorporating helix-terminators (e.g., proline residues).

Tertiary (three-dimensional) protein 210 structure can be predicted using a variety of known molecular modeling techniques, including but not limited to Monte Carlo simulation (e.g., Sadanobu and Goddard, J. Chem. Phys. 106:6722, 1997), energy minimization, molecular dynamics (e.g., van Gunsteren and Berendsen, Angew. Chem. Int. Ed. Engl. 29:992-1023, 1990), topomer sampling methods (e.g., Debe et al., Proc. Nat. Acad. Sci. USA, 96:2596-2601, 1999) and other known methods. Standard computer modeling programs for prediction of protein 210 tertiary structure are available (e.g., AMBER, http://www.amber.ucsf.edu/amber; X-PLOR, Yale University, New Haven, Conn.; INSIGHTII, Molecular Simulations Inc., San Diego, Calif.; CHARMM, Harvard University, Cambridge, Mass.; DISCOVER, Molecular Simulations Inc., San Diego, Calif.; GROMOS, ETH Zurich, Zurich, Switzerland).

Various exemplary databases containing protein 210 structural information and/or computer programs for predicting protein 210 structure are shown in Table 1 below. (See also http://www.aber.ac.uk/˜phiwww/prof; http://www.embl-heidelberg.de/cgi/predator_{—}serv.pl; http://www.embl-heidelberg.de/predictprotein/ppDoPredDef.html).

TABLE 1
Protein Structure Databases

| Database | Web Sites |
|---|---|
| FASTA | ebi.ac.uk/fasta3 (world-wide web 2) |
| BLAST | ncbi.nlm.nih.gov/BLAST/(world-wide web) |
| | ebi.ac.uk/blast2 (world-wide web) |
| Clustal W | ebi.ac.uk/clustal (world-wide web 2) |
| AMAS | barton.ebi.ac.uk/servers/amas_server.html |
| | (Internet) |
| PDB | rcsb.org (world-wide web) |
| PROCHECK | biochem.ucl.ac.uk/~roman/procheck/procheck.html |
| | (world-wideweb) |
| COMPOSER | cryst.bioc.cam.ac.uk (internet) |
| MODELLER | guitar.Rockefeller.edu/modeler.html (internet |
| SWISS- | expasy.ch/swissmod/SWISS-MODEL.html (world-wide |
| MODEL | web) |
| SCOP | scop.mrc-lmb.cam.ac.uk./scop (Internet) |
| CATH | biochem.ucl.ac.uk/bsm/cath (world-wide web) |
| FSSP | ebi.ac.uk/dali/fssp.html (world-wide web) |
| MMDB | ncbi.nlm.nih.gov/Structure/MMDB/mmdb/html (world- |
| | wide web) |
| THREADER | insulin.brunel.ac.uk/threader/threader.html |
| | (Internet) |
| TOPITS | embl-heidelberg.de/predictprotein/ppDoPredDef.html |
| | (world-wideweb) |
| CASP | predictioncenter.llnl.gov/casp2/Casp2.html |
| | (Internet) |
| | predictioncenter.llnl.gov/casp3 (Internet) |

Methods of designing protein 210 sequences capable of forming quaternary assemblies of proteins 210 are known in the art. For example, Aggeli et al. (2001) disclosed an anti-parallel p-sheet structure, based upon 11 amino acid residue rod-like monomers 210, capable of one-dimensional self-assembly in solution to form regular arrays of tertiary structures, referred to as tapes, ribbons, fibrils and fibers. The 8 nm wide fibrils were observed to be extremely stable. Because the monomers 210 were designed to have different upper and lower surfaces (e.g. hydrophilic and hydrophobic), self-assembly of such a structure on a silicon substrate should result in an ordered, two-dimensional array of regularly repeating subunits 210. The rod-like monomer 210 structures disclosed by Aggeli et al. (2001) exhibited an inherent chirality due to the chiral nature of L-amino acids, resulting in twisting of the tertiary structure. In applications where twisting is undesirable, use of alternating L- and D-amino acids can eliminate the monomer 210 chirality and improve the stability of a planar assembly of monomers 210.

In another non-limiting example, Brown et al. (2002) discussed the template-directed assembly of a de novo designed protein 210, composed of 63-amino acid residue monomers 210 designed to assemble into an antiparallel β-sheet. The monomers 210 were comprised of 6 β-strands, each of 7 amino acids in length. The two sides of the sheet were designed to be either highly hydrophobic or highly hydrophilic. A monomeric solution of protein 210 was exposed to a highly ordered pyrolytic graphite (HOPG) surface, comprising a hexagonal array of crystals. The results showed that the monomers 210 assembled into a sheet-like structure coating the HOPG surface, with different portions of the structure exhibiting three preferred orientations at 1200 to each other. It was proposed that the 3-fold symmetry of the assembled proteins 210 was imposed by the hexagonal structure of the underlying graphite. Deposit of protein 210 on an amorphous carbon surface did not result in ordered arrays of proteins 210. Such an assemblage of proteins 210 can be used to coat areas 110, 310 of a substrate, such as a silicon chip. Because the underlying silicon is not hexagonal in structure, it is expected that the protein 210 assembly would exhibit a 2-fold rather than a 3-fold symmetry.

These and other known methods for attaching protein monomers 210 to a substrate in an ordered array can be used in the methods and apparatus disclosed herein. Naturally occurring proteins 210, such as viral coat proteins 210, that spontaneously assemble into ordered arrays can be used. Alternatively, synthetic proteins 210 designed to assemble into ordered arrays can be purchased or chemically synthesized. Synthetic proteins 210 can be produced with modified amino acid residues (e.g. biocytin 220) or amino acid analogues incorporated at specific locations in the primary and tertiary structures of the protein 210. Naturally occurring proteins 210 can be chemically modified using known side-chain specific reagents (e.g., Bell and Bell, Proteins and Enzymes, Ch. 7 and 8, Prentice-Hall, Inc., Englewood Cliffs, N.J. 1988). In either case, catalyst nanoparticles 140, 230 can be attached to the proteins 210 at selected locations, for example using binding between biotin 160 and avidin 170 moieties as discussed above. Alternatively, catalyst nanoparticles 140, 230 could be attached to antibodies or antibody fragments that bind to specific locations on protein monomers 210. In other alternatives, nucleic acid 120 sequences could be attached to proteins 210 at selected locations and hybridized to oligonucleotides 150 containing attached catalyst nanoparticles 140, 230.

Proteins 210 can be aligned using any known molecular alignment method, such as molecular combing, optical tweezers, microfluidic flow, magnetic fields, free flow electrophoresis, etc., as discussed above. Proteins 210 can be attached to substrates using standard techniques, such as silanization and activation via carbodiimide or glutaraldehyde. Alternative procedures can use reagents such as 3-glycidoxypropyltrimethoxysilane (GOP) or aminopropyltrimethoxysilane (APTS) linked via amino groups. Other known methods, such as forming micropatterned mercaptobenzoic acid and/or mercaptohexadecanoic acid monolayers on gold patches 110, 310 (e.g. Liu and Amro, Proc. Natl. Acad. Sci. USA, 99:5165-70, 2002) can be used. In this case, the mercapto moieties bind to the gold patches 110, 310, allowing attachment of proteins 210, for example by carbodiimide catalyzed covalent bond formation between acidic moieties on the monolayer and terminal or side-chain amino groups. Alternatively, acid-acid dimer hydrogen bonding can occur between carboxyl groups on the monolayer and protein 210. Proteins can also be immobilized on gold patches 110, 310 using self-assembling monolayers (SAM) of 4-mercaptobenzoic acid. In other alternative embodiments of the invention, gold binding proteins 210 (e.g. Brown, Nano Lett. 1:391-394, 2001) can be used to directly attach proteins 210 to gold patches 110, 310. The methods are not limiting and any known procedure for attaching and/or aligning protein molecules 210 on substrates can be used.

In particular embodiments of the invention, protein monomers 210 can be ligated together, for example to form concatemers and/or chains of proteins 210. Methods of protein 210 ligation and concatenation are generally known (e.g., Thompson and Ellman, Chem. Rev. 96:555-600, 1996; Cotton and Muir, Chemistry & Biology 6:R247, 1999; Nilsson et al., Organic Lett. 2:1939, 2000) and any such known method can be used.

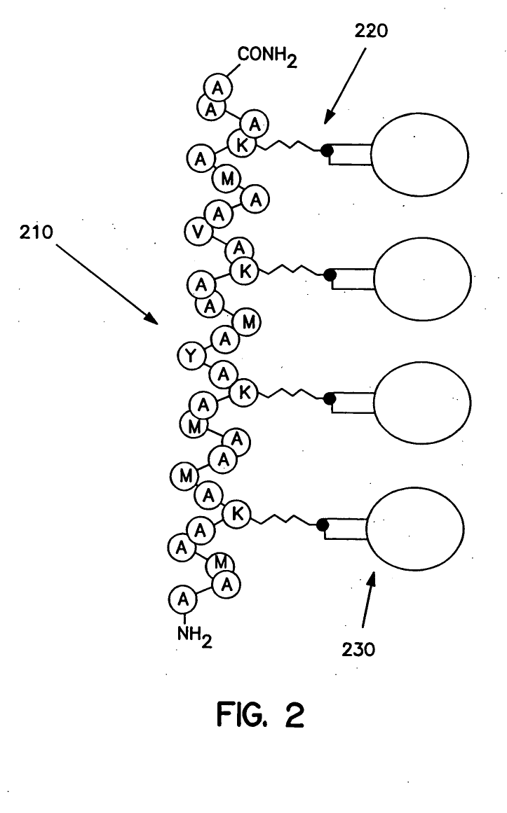

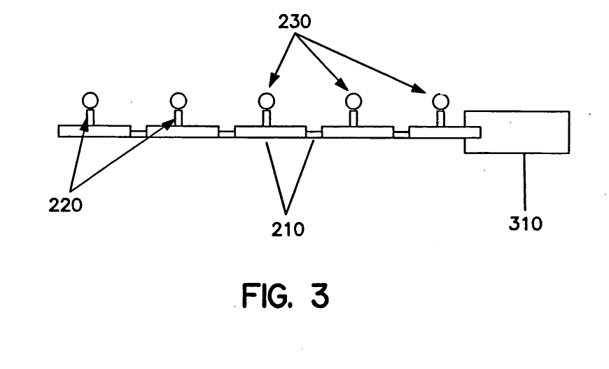

An exemplary embodiment of the invention illustrating a method for producing patterned arrays of carbon nanotubes using proteins 120 attached to a substrate, is disclosed in FIG. 2 and FIG. 3.

FIG. 2 shows an exemplary protein 210, comprising a linear polymer 210 of amino acids, amino acid analogues and/or modified amino acids. In this non-limiting example, certain lysine residues have been substituted with biocytin 220, a biotinylated form of lysine. In this case, the protein 210 can be produced by chemical synthesis, incorporating biocytin 220 residues during the synthetic process. Alternatively, a synthetic or naturally occurring protein 210 or protein 210 can be chemically modified to attach biotin 160 or other nanoparticle 230 binding groups after synthesis or post-translationally. Where a synthetic protein 210 is used, the protein 210 sequence can be designed to form specific secondary, tertiary and/or quaternary structures, using known methods (e.g., Aggeli et al., 2001; Brown et al., 2002). For example, the synthetic protein 210 disclosed in Brown et al. (2002) contained a number of lysine residues, one or more of which could be substituted by biocytin 220. Because such residues are on the hydrophilic face of the β-sheet structure formed by that protein 210, the biotin moieties 160 would be exposed to the aqueous medium where they could bind to avidin 170 conjugated ferritin nanoparticles 230. The protein 210 of Brown et al. (2002) has been demonstrated to assemble into ordered arrays on a HOPG surface and could be used to coat selected areas 310 on a substrate, such as a silicon chip. In alternative embodiments of the invention, monomeric proteins 210 could potentially be ligated into chains or concatemers of proteins 210, using known techniques.

In an exemplary embodiment of the invention, the synthetic protein 210 could be attached to the substrate, for example by incorporating a terminal cysteine residue and attaching the sulfhydryl group to a gold monolayer coated onto selected areas 310 of the substrate. Alternatively, micropatterned mercaptobenzoic acid and/or mercaptohexadecanoic acid monolayers could be covalently bound to a gold layer coated onto selected areas 310 of a substrate. The terminal acidic groups could be covalently attached to terminal or side-chain amino groups on the protein 210, for example using a water-soluble carbodiimide. The examples are not limiting and any method of attaching proteins 210 to a substrate can be used. To check the number and pattern of proteins 210 attached to the substrate, dye-stained proteins 210 could be visualized by fluorescence microscopy. Alternatively, nanoparticle 230 conjugated proteins 210 could be visualized by SPM techniques, such as atomic force microscopy (AFM) or scanning tunneling microscopy (STM).

FIG. 3 illustrates an exemplary nanoparticle 230 conjugated protein 210 attached to a substrate. For example, a terminal cysteine residue could be covalently bound to gold-coated areas 310 on the substrate. The attached protein 210 can be aligned by any known molecular alignment technique, such as optical tweezers, electrophoresis, magnetic fields, molecular combing, microfluidic flow, etc. After alignment, the proteins 210 can be immobilized on the substrate, for example, by drying.

Catalyst nanoparticles 230 can be attached to the proteins 210 before or after the proteins 210 are attached to the substrate. In embodiments of the invention where the proteins 210 self-assemble on the substrate, it can be beneficial to attach the nanoparticles 230 after the protein 210 array has been formed. In this non-limiting example, avidin 170 conjugated ferritin nanoparticles 230 can be exposed to biocytin groups 220 on the proteins 210. A one-to-one binding between avidin 170 and biocytin 220 occurs, resulting in each biocytin residue 220 attaching to one ferritin nanoparticle 230. This would result in an ordered array of catalyst nanoparticles 230 arranged on the selected areas 310 of the substrate. After the substrate is washed and dried to remove unbound nanoparticles 230, carbon nanotubes can be formed by CVD methods as disclosed above. The remaining proteins 210 and the ferritin component of the nanoparticles 230 can be removed by heating in air or oxygen as disclosed above, leaving a substrate attached to an ordered array of carbon nanotubes. Because the proteins 210 can pack into a highly ordered array on the substrate, with nanoparticles 230 attached at regularly repeating intervals, both the distance between adjacent nanotubes and the pattern of nanotubes arrayed within each area 310 can be determined.

Although the invention has been described above, it will be understood that modifications and variations are encompassed within the spirit and scope of the invention. Accordingly, the invention is limited only by the following claims.

1-30. (canceled)

31. A method for aligning a molecular wire, comprising:
a) ligating the molecular wire to a double stranded DNA molecule to create a double-stranded DNA/molecular wire hybrid molecule;
b) applying the double-stranded DNA/molecular wire hybrid to an anchor surface; and
c) aligning the double-stranded DNA/molecular wire hybrid to the anchor surface using fluidic alignment.

32. The method of claim 31, further comprising drying the double-stranded DNA/molecular wire hybrid molecule to the surface.

33. The method of claim 32, wherein the molecular wire is a single-stranded nucleic acid.

34. The method of claim 33, wherein the singe-stranded nucleic acid is single-stranded DNA.

35. The method of claim 32, wherein the molecular wire is attached to a catalytic nanoparticle.

36. The method of claim 35, further comprising producing carbon nanotubes from the catalyst nanoparticles.

37. The method of claim 33, wherein the double-stranded DNA is phage lambda DNA.

38. The method of claim 33, further comprising hybridizing an oligonucleotide to the single-stranded nucleic acid.
